(19) United States
(12) Patent Application Publication
(10) Pub. No.: US 2009/0028954 A1
(43) Pub. Date: Jan. 29, 2009

(54) PRECURSOR FOR THE PREPARATION OF A PASTY BONE REPLACEMENT MATERIAL BY ADMIXTURE OF A LIQUID

(75) Inventors: Marc Bohner, Grenchen (CH); Giancarlo Rizzoli, Bern (CH)

Correspondence Address: RANKIN, HILL & CLARK LLP, 38210 Glenn Avenue, WILLOUGHBY, OH, 44094-7808, US

(73) Assignee: MATHYS AG BETTLACH, Bettlach (CH)

(21) Appl. No.: 12/280,094

(22) PCT Filed: Dec. 22, 2006
(86) PCT No.: PCT/CH2006/000736

§ 371 Date: Oct. 6, 2008

(51) Int. Cl. A61K 9/14

(52) U.S. Cl. 424/489

(57) ABSTRACT

The precursor is used for the preparation of a pasty bone replacement material by admixture of a liquid. The precursor comprises a biocompatible substance swellable by the action of water or of an aqueous solution thereby forming a hydrogel; and solid particles made of a substance which is suitable as a bone replacement material. The swellable substance is in the form of discrete particles having a mean dimension in the range of 18 μm to 2000 μm.

### FIELD OF THE INVENTION

The invention relates to a precursor for the preparation of a pasty bone replacement material by admixture of a liquid according to the preamble of claim 1.

### DESCRIPTION OF THE PRIOR ART

A number of bone replacement material prepared from solid, dry precursors by admixing of a liquid are known. However, all of the known precursor materials are either non-sterile or are degraded in their molecular structure by the sterilization process. In particular the usual dry autoclaving (e.g. for 120 minutes at 170° C.—WHO 1986) leads to the destruction of most hydrogels used in such bone replacement materials.

Materials which can be injected are also known. For example, hydraulic calcium phosphate cements consist of one or several calcium phosphate powders and an aqueous solution. Upon mixing, a paste is formed. This paste can be injected and hardens within (typically) 5-20 minutes. Unfortunately, the resulting hardened paste is still brittle and can only be resorbed layer-by-layer, i.e. much slower than the pastes described in the present invention. Other injectable pastes consist of non cementitious mixtures of microsized calcium phosphate particles and an aqueous solution. Again, resorption only occurs layer-by-layer. A third alternative is to combine spherical particles (larger than about 0.1 mm) and a low-viscosity hydrogel. These mixtures are injectable, and have a well distributed resorption due to the presence of gaps between the spherical particles, but these mixtures are not kneadable and present a low cohesion.

In the following text, the term “particle” includes any three-dimensional body, regardless of its dimensions, especially the small parts commonly known as “granules” or “grains”. The sphericity S of the particles (or spherical relationship) is defined as the ratio of Dₘₐₓ/Dₘᵢₙ between the largest diameter Dₘₐₓ and the smallest diameter Dₘᵢₙ of the individual particles. Fully spherical particles therefore have a sphericity S=1.00.

This discussion regarding current standards of technology is used only to explain the environment of the invention and does not mean that the standards of technology quoted here were actually published or publicly known at the time of this registration or its priority.

This invention is meant to provide a remedy for this situation. The invention is based on the problem of creating a precursor which overcomes the disadvantages listed above. The invention solves this task with a precursor which has the characteristics of claim 1.

It is an object of the invention to provide a solid precursor for the preparation of a pasty bone replacement material by admixture of a liquid, whereby said precursor remains stable prior to use and in particular retains its molecular integrity to a high degree.

The advantages of the invention are the following:
Large versatility since the dry precursor can be mixed with many different liquids such as blood, platelet-rich plasma, antibiotic solution or bone marrow;
Very good handling (fast obtention of a pasty material);
Optimum resorption of the kneadable bone replacement material obtained from the precursor;
good optical appearance (no color heterogeneities due to the presence of solid particles having a inadequate size); and
easy production (due to a sufficiently large size of the solid particles allowing automatic weighing.

In a special embodiment the precursor has been obtained by wet autoclaving and subsequent drying of at least said swellable substance.

A hydrogel is present when a solid substance is hydrated via a liquid phase, changing and increasing the viscosity of the liquid phase, i.e. jellying or coagulating the liquid phase. Some hydrogels are rather elastic others are rather plastic (e.g. sodium hyaluronate). An elastic hydrogel can be destroyed with shear forces, contrary to a plastic (deformable) hydrogel.

The said solid particles may be of ceramic or mineral nature and they may contain calcium. The solid particles may also be based on demineralized or purified bone material. The demineralized or purified bone material can be obtained either from natural bone by extracting all organics from bovine bone.

The solid particles may alternative by of polymeric nature, preferably of polylactic acid (PLA) or polyglycolic acid (PGA) or they may be based on bioglass(es). The hydrogel matrix can consist of oligomeric or polymeric parts or of a combination of the two.

The solid particles and said swellable substance may be present as a mixture. Alternatively the swellable substance is in powdered form.

In a further embodiment the discrete particles of said swellable substance have a mean diameter of at least 18 μm, preferably at least 50 μm. In a further embodiment the discrete particles of said swellable substance have a mean diameter of less than 2000 μm, preferably less than 1000 μm.

The swellable substance may contain a polyamino-acid or its derivatives, preferably polylysin or gelatin. The swellable substance may alternatively contain one of the following components: a) polysaccharides and their derivatives, preferably glycosaminoglycane or alginate; b) polylipides, fatty acids and their derivatives; c) nucleotides and their derivatives; d) polymethylenoxide or its derivatives; e) polyethylene, polyethylenoxide or their derivatives; f) polypropylene, polypropylenoxide or their derivatives; g) polyacrylate or its derivatives; or a combination of the components as listed in a) through g).

The swellable substance may consist of either a glycosaminoglycane or a proteoglycane or a mixture of those two substances. The glycosaminoglycane may be a hyaluronic acid, chondroitinsulfate, dermatansulfate, heparansulfate, heparine or keratansulfate.

In a further embodiment the hydrogel is hyaluronic acid. The hyaluronic acid consists of glucuronic acid and acetylglucosamine which create the disaccharide hyaluronic acid. The hyaluronic acid has a fibrous, non-branched molecular structure and therefore results in highly viscous liquid solutions. The hydrogel may also be in the form of sodium hyaluronate (NaHyA).

In a further embodiment the swellable substance is of fully synthetic origin. This eliminates the danger of transferring diseases due to the absence of possible pathogenic agents such as proteins, germs, viruses or bacteria as compared to precursors of natural origin.

Alternatively the swellable substance may consist of a biotechnologically generated substance (e.g. fermentation).

In a further embodiment the MW of the swellable substance is—after sterilization—larger than 300'000 Dalton and preferably larger than 500'000 Dalton. In a further embodiment the MW of the swellable substance is larger than 1'000'000 Dalton and preferably larger than 1'500'000 Dalton. Concerning the MW, it is important to know that it is often calculated based on viscosity data, in particular the “intrinsic viscosity” of the polymer. It may be advantageous therefore to specify the intrinsic viscosity of the polymer rather than its MW. An initial intrinsic viscosity larger than 2.0 m³/kg, preferably larger than 2.5 m³/kg appears to be the minimum required to obtain a good product after sterilization. After sterilization, the intrinsic viscosity should preferably be superior to 1.3 m³/kg.

In a further embodiment said swellable substance has an intrinsic viscosity of at least 1.3 m³/kg, preferably at least 1.4 m³/kg after sterilization. Preferably at least 80% of said intrinsic viscosity is reached within 5 minutes, preferably within 2 minutes after the start of mixing.

In a further embodiment said discrete particles of said swellable substance have a sphericity S smaller than 5, preferably smaller than 2.

In a further embodiment the precursor further comprises a drug having an active effect on bone metabolism, preferably osteoinductive substances, drugs against osteoporosis or antimicrobial drugs. Examples for osteoinductive substances are: morphogenetic proteins and growth factors; examples for drugs against osteoporosis are: biphosphonates and parathyroid hormone; an example for an antimicrobial drug is gentamycin sulfate.

In a further embodiment the solid particles have at least a partially porous structure. The pore size of the solid particles is preferably between 10 nanometers and 500 micrometers.

Preferably at least 50% of the solid particles have a pore size between 100 and 500 micrometers. This guarantees optimum pore size distribution and the growth of autogenous tissue into the pores.

In a further embodiment the porosity of the solid particles is between 60 and 90 percent, preferably between 68 and 84 percent. This ensures that autogenous tissue is able to grow into a large volume share of solid particles.

The average diameter of the solid particles is preferably between 100 and 500 micrometers. The advantage of this is the fact that the paste obtained by admixing the precursor with a liquid gets a smooth consistency. In addition, the risk of irritation within the tissue surrounding the particles is practically non-existent, if the diameter of the particles is not smaller than 100 micrometers.

It is also possible to mix the solid particles with two different size populations, e.g. particles with an average diameter between 125 and 250 micrometers and particles with an average diameter between 500 and 710 micrometers or an average diameter between 0.5 and 5.6 mm. This has the advantage that it guarantees the compactness of the bone replacement material. The interstitial pore volume (pore dead volume) which results from the use of large-grain material can thus be reduced to a minimum. It is also possible to affect the degradation period of the bone replacement material through the use of solid particles of various sizes. (smaller particles are resorbed faster than larger particles).

The specific gravity of said solid particles may be between 0.5 and 1.0 g/ccm. Alternatively to the spherical form the solid particles may have also a non-spherical shape. The solid particles may have a specific surface area (SSA) of larger than 0.01 m²/g, preferably larger than 0.1 m²/g. The solid particles may have also a specific surface area (SSA) of larger than 5 m²/g, preferably larger than 50 m²/g. A high SSA is advantageous for drug delivery purposes. Drugs trapped in the porous structure diffuse out at a very slow rate, typically over days or even weeks.

In a special embodiment the discrete particles of said swellable substance have a mean volume of at least 3·10⁻⁶ mm³, preferably of at least 65·10⁻⁶ mm³. In another embodiment the discrete particles of said swellable substance have a mean volume of maximum 4.2 mm³, preferably of maximum 0.5 mm³.

In a further embodiment the precursor contains less than 10 weight-percent, preferably less than 1 weight-percent of gelatin. Most preferably the precursor is free of gelatin.

In a further embodiment the precursor contains a radiopacifier. The use of a radiopacifier is beneficial for certain application, such as vertebroplasty or kyphoplasty. It allows a better visualization of the position of the bone substitute during and after insertion into the bone defect. The radiopacifier may be selected from the following group: tantalum powder, tungsten powder, titanium powder, zirconium oxide powder, bismuth oxide powder, iode-based liquid. A suitable iode-based liquid is iopamidol.

To improve the biological efficiency, the paste produced by mixing the various components of the precursor according to the invention should preferably present large domains between the solid particles to allow a rapid cell invasion through the hydrogel (present between the solid particles) and hence enable a fast ingrowth of bone within the bone substitute (because the hydrogel is resorbed or removed within a few days).

The term “Non-spherical” describes any particle shape which is significantly different from a spherical shape. One variant of the invention uses solid particles with an angular shape. “Angular” describes those particles which have individual edges, especially those which are visible with the naked eye, i.e. which are at least 0.1 mm in size. Compared to round particles, these results in an increase to the particle surface, while the average particle diameter remains the same. This causes the adhesive interaction between the solid particles and the hydrogel to be increased, guaranteeing the moldability of the bone-replacement material without the need for increasing the quantity of hydrogel used or its concentration.

There is also a special embodiment where the solid particles have a spherical relationship S=Dmax/Dmin between the largest diameter Dmax and the smallest diameter Dmin of the individual particles, which is larger than 1.2 and preferably larger than 1.5. The value of S should be larger than 3 and preferably larger than 5. Preferably at least 60% and typically at least 80% of the solid particles should be of a non-spherical shape.

Alternatively, it might be of interest to provide an injectable paste. For that purpose, it is important to use round particles. Mixtures of various particle sizes can lead to a more compact paste that is also better injectable.

To provide a fast bone ingrowth and resorption of the solid particles, at least 60% and typically at least 80% of the solid particles should be of a non-spherical shape.

In a further embodiment the packed bulk density of the solid particles, e.g. in form of calcium containing, porous ceramic particles is preferably between 0.5 and 1.0 g/ccm.

Particles with a high specific surface area (SSA) are characterized by the presence of numerous nanosized pores or by a high surface corrugation. As a result, such particles are of great interest for drug delivery applications: drugs entrapped in the nanopores diffuse at a very low rate, hence providing an excellent drug delivery system. Particles obtained at high temperature (e.g. β-tricalcium phosphate, α-tricalcium phosphate, tetracalcium phosphate, calcined bone) have normally a very low SSA, typically in the range of 0.001 to 1 m²/g. Particles synthesized at or close to room temperature present generally much higher SSA. For example, particles obtained by purifying bone chips (extraction of organic matter) have a SSA typically in the range of 50-100 m²/g. Particles obtained by hydraulic reactions (e.g. calcium phosphate cement) have a SSA in the range of 10-200 m²/g depending on the composition.

In a further embodiment the solid particles comprise a calcium phosphate which is characterized by a molar Ca/P relationship between 1.0 and 2.0. Preferably the ceramic particles comprise a calcium phosphate which is characterized by a molar Ca/P relationship between 1.45 and 1.52.

The calcium phosphate may be selected from the following group: Dicalcium phosphate dihydrate (CaHPO₄×2H₂O), dicalcium phosphate (CaHPO₄), alpha-tricalcium phosphate (alpha-Ca₃(PO₄)₂), β-tricalcium phosphate (β—Ca₃(PO₄)₂), calcium-deficient hydroxyapatite (Ca₉(PO₄)₅(HPO₄)OH), hydroxyapatite (Ca₁₀(PO₄)₆OH)₂), carbonated apatite (Ca₁₀(PO₄)₃(CO₃)₃(OH)₂), fluoro apatite (Ca₁₀(PO₄)₆(F,OH)₂), chloro apatite (Ca₁₀(PO₄)₆(CI,OH)₂), whitlockite ((Ca,Mg)₃(PO₄)₂), tetracalcium phosphate (Ca₄(PO₄)₂O), oxyapatite (Ca₁₀(PO₄)₆O), β-calcium pyrophosphate (β—Ca₂(P₂O₇), α-calcium pyrophosphate, gamma-calcium pyrophosphate, octacalcium phosphate (Ca₈H₂(PO₄)₆×5H₂O). The various calcium phosphate materials may also be doped with elements such as Na, Cl, F, S, C, Sr, Mg, Zn, Si, Fe, Li, K or Ag.

In a further embodiment the solid particles comprise a mixture of different calcium phosphates. The advantage of such a mixture lies in the control of the resorption period. Due to the differing resorption behaviors of the mixture components, faster bone growth into the cavities of components with faster resorption times can be facilitated.

Alternatively the solid particles may comprise a calcium sulphate (anhydrous, hemihydrate, dehydrate, and their polymorphs), a calcium carbonate (calcite, aragonite or vaterite), bioglass or a mixture of different calcium phosphates, calcium sulfates, calcium carbonates and/or calcium-containing bioglass. The advantage of such a mixture lies in the control of the resorption period. Due to the differing resorption behaviors of the mixture components, faster bone growth into the cavities of components with faster resorption times can be facilitated.

Preferably, the maximum amount of residual water present in the solid precursor (expressed by the loss on drying at 105° C.) is smaller than 5%, preferably smaller than 2%. The presence of absorbed water triggers the decomposition of the hydrogel and hence the residual humidity in the dry product should be kept as low as possible.

The preparation of a bone replacement material is obtained by admixing a liquid to the precursor. The following liquids are suitable for that purpose: pure water, sterile demineralized water, an aqueous solution, a sterile saline solution, sterile Ringer solution, an antimicrobial drug solution preferably an antibiotic solution—or a solution containing osteoinductive substances—preferably bone morphogenetic proteins such as BMP2 and BMP7 or growth factors—and/or drugs against osteoporosis—preferably bisphosphonates and parathyroid hormone. The surgeon has the possibility to replace the sterile solution with blood, blood extract (e.g. serum, platelet rich plasma), bone marrow, bone marrow extract, or any human extract having a beneficial effect on bone formation.

The solid precursor should be sterile for surgical use. There are two sterilization approaches that can be used: (i) combine two sterile products and package them in an aseptic environment, or (ii) prepare the solid precursor and then sterilize it. The first approach is at first sight the easiest, but for production cost reasons, the second approach is nowadays the best approach. Therefore, the bone replacement material and the swellable substance must be simultaneously sterilized. Among the various sterilization techniques that can be used for solids (gamma irradiation, plasma, ethylene oxide, autoclaving, hot air), autoclaving is the best possible technique due to (i) the good homogeneity of the sterilization method, (ii) an absence of toxicity, and (iii) the ability to retain the molecular integrity of the powder substance.

Preferably, autoclaving (=steam sterilization) is done in such a way that it does not lead to a molecular weight (MW) loss of the hydrogel greater than 70%. Autoclaving may be performed at various temperatures for various durations. In fact, higher temperatures require shorter sterilization times (see Chapter 4 “Verfahren zur Verminderung der Keimzahl” of “Sterilisation, Desinfektion, Konservierung, Keimidentifizierung, Betriebshygiene (edition 1988).

Keeping a given autoclaving duration (e.g. 18 min), a too low temperature is not able to sterilize the sample, whereas a too large temperature drastically destroys the polymer. As a result, an intermediate temperature is ideal. For example, it could be observed with sodium hyaluronate and β-TCP granule mixtures that a temperature of 121° C. (18 min autoclaving) was too low (only partly sterile) whereas a temperature of 128° C. (18 min autoclaving) was too high. A temperature of 125° C. for a duration of 18 min appeared therefore to be optimal.

Preferably the autoclaving does lead to a decrease of the MW of said swellable substance of minimum 30% and of maximum 70%. The autoclaving may be performed at a temperature in the range of 110° to 140° C., preferably of 121°-128° C. The drying of the precursor may be obtained by the action of dry air, vacuum, freeze-drying and/or a desiccating agent. Preferably the loss on drying at 105° C. of said precursor is smaller than 5%, preferably smaller than 2%.

In a preferred embodiment the ratio between the dry weight of the swellable substance and the liquid is in the range of 0.001 and 0.500. Higher concentrations lead to higher costs and lower concentrations do not lead to the desired plastic and firm type material. Preferably the ratio between the dry weight of the swellable substance and the liquid is in the range of 0.03 and 0.09.

In a further embodiment the weight relationship between the hydrated hydrogel and the solid particles is larger than 0.2, preferably larger than 0.6. In another embodiment the weight relationship between the hydrated hydrogel and the solid particles is smaller than 4, preferably smaller than 2.

The precursor can be made available in form of a kit comprising the precursor together with a liquid suitable for admixing to said precursor in order to convert the resulting mixture into a kneadable mass for bone replacement. Preferably the bone replacement material product is presented to the surgeon as a kit consisting of a sterile powder [e.g. β-TCP granules+NaHyA powder] and a sterile liquid, e.g. deionized water or saline solution.

Experimentally, so-called cohesion tests have been performed with pastes produced with various NaHyA particle sizes. The “cohesion” of a paste is defined as the ability of a paste to stay in one piece when placed into contact with an aqueous solution. This property can be measured by dipping a paste in an aqueous solution (e.g. 4 minutes after the start of the preparation) and measuring the weight loss of the paste over time (Bohner et al, Eur Cells Mater, 2006). Two approaches can be used to quantify the results: measure how long it takes until a known amount of weight has been lost (e.g. −0.3 g) or how much material has been lost within a given time period (e.g. between the time points 10 and 30 minutes of the measurement)

The precursor according to the invention has to be mixed with a liquid to obtain a pasty material. It has been found that the viscosity of the resulting paste is not only a function of the concentration and molecular weight of the swellable substance, but also of the kinetics of the dissolution of the swellable substance.

Since the dissolution of the swellable substance is a function of the interface area between the swellable substance and liquid, small particles dissolve much faster than large particles. As a result, relatively small particles are preferred to large particles.

Small particles do not flow very well. Therefore, big particles should be preferred to small particles for production purposes because it is easier to automatically weigh them. Similarly, round particles flow much better than fibers, i.e. round and large particles (>20-50 microns in diameter) are the most adapted to production purposes.

Furthermore, the particles of the swellable substance tend to shrink during autoclaving and subsequent drying. The resulting change of particle density may provoke a change of the optical appearance of the hydrogel. For example, the color of NaHyA particles changes from white/translucent to yellow. As calcium phosphate particles are generally white, the presence of yellow particles in the product has a negative effect on its aesthetic properties. This effect is a function of the hydrogel particle size: particles smaller than about 0.5 mm are too small to be detected by eye sight, and hence the product appearance is maintained (no apparent heterogeneities in the product), i.e. the hydrogel particles should have a diameter smaller than 1 mm, preferably smaller than 0.5 mm.

### A BRIEF DESCRIPTION OF THE DRAWINGS

FIG. 1: representative curves obtained in the cohesion test with a 1.76 MDa NaHyA powder (intrinsic viscosity: 2.68 m³/kg). The particle diameter was: (▴) d<0.125 mm; (∘) 0.125<d<0.250 mm; (x) 0.250<d<0.500 mm; (Δ) 0.500<d<1.000 mm. Putty composition: 0.94 g 0.125-0.250 mm β-TCP granules, 0.94 g 0.50-0.71 mm β-TCP granules, 102 mg NaHyA, 1.6 mL deionized water.

FIG. 2: representative curves obtained in the cohesion test with a 1.1 MDa NaHyA powder (intrinsic viscosity: 1.64 m³/kg). The particle diameter was: (▴) d<0.125 mm; (∘) 0.125<d<0.250 mm; (x) 0.250<d<0.500 mm; (A) 0.500<d<1.000 mm; and () 1.0<d<2.0 mm. Putty composition: 0.94 g 0.125-0.250 mm β-TCP granules, 0.94 g 0.50-0.71 mm β-TCP granules, 102 mg NaHyA, 1.6 mL deionized water.

FIG. 3: Effect of particle size on the average duration until the weight loss amounted −0.3 g; (x) 1.76 MDa powder (intrinsic viscosity: 2.68 m³/kg); (Δ) 1.1 MDa powder (Intrinsic viscosity: 1.64 m³/kg).

FIG. 4: Effect of particle size on the weight loss between 10 and 30 min; (x) 1.76 MDa powder (intrinsic viscosity: 2.68 m³/kg); (A) 1.1 MDa powder (Intrinsic viscosity: 1.64 m³/kg).

FIG. 5: Effect of NaHyA molecular weight, particle size and sterilization on the duration until a 0.3 g weight loss was measured during cohesion testing. (∘) 1.76 MDa NaHyA, before autoclaving; () 1.76 MDa NaHyA, after autoclaving; (Δ) 1.1 MDa NaHyA, before autoclaving; (A) 1.1 MDa NaHyA, after autoclaving (only one measurement). The red dotted line represents the value obtained with a 0.67 MDa NaHyA powder (intrinsic viscosity: 1.3 m³/kg).

FIG. 6: Effect of NaHyA particle size and sterilization on the cohesion weight loss measured between 10 and 30 min of testing. (∘) 1.76 MDa NaHyA, before autoclaving; () 1.76 MDa NaHyA, after autoclaving; (A) 1.1 MDa NaHyA, before autoclaving. The red dotted line represents the value obtained with a 0.67 MDa NaHyA powder (intrinsic viscosity: 1.3 m³/kg). No values were reported for 1.1 MDa NaHyA powder after autoclaving due to the fact that the paste fell off within the first 10 minutes.

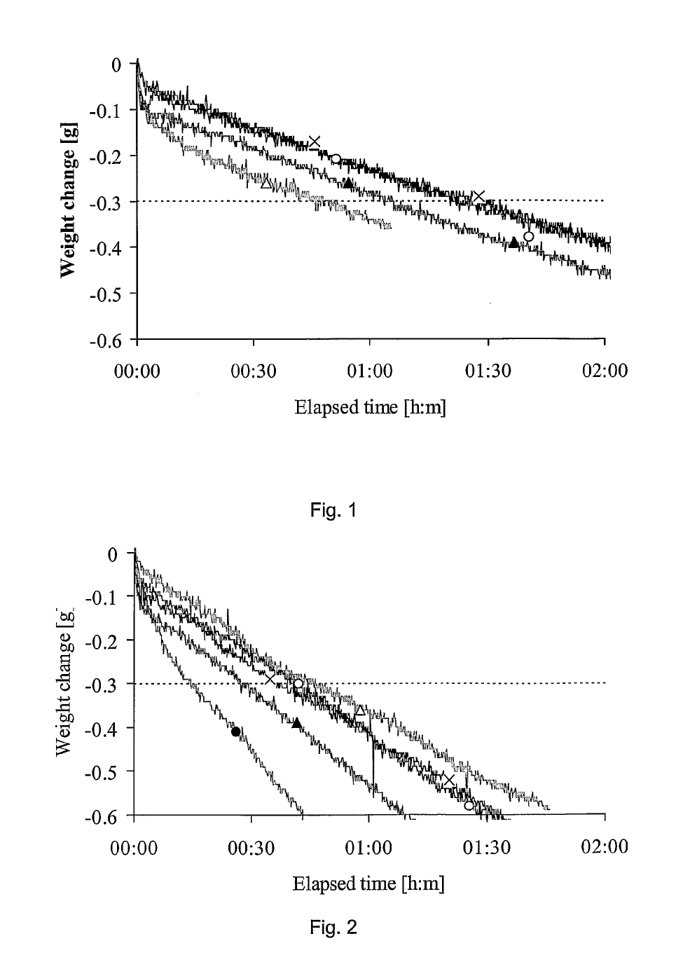

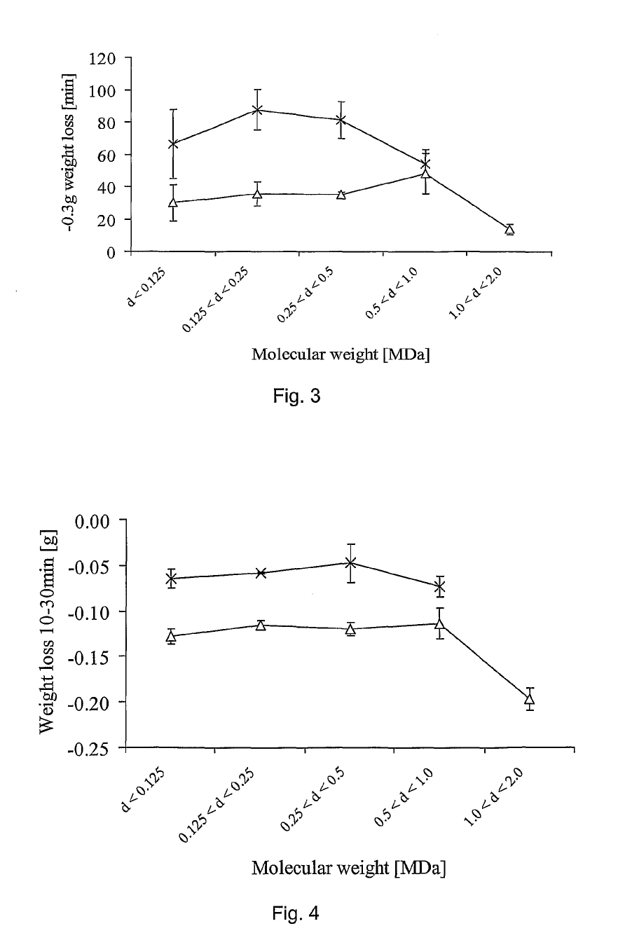

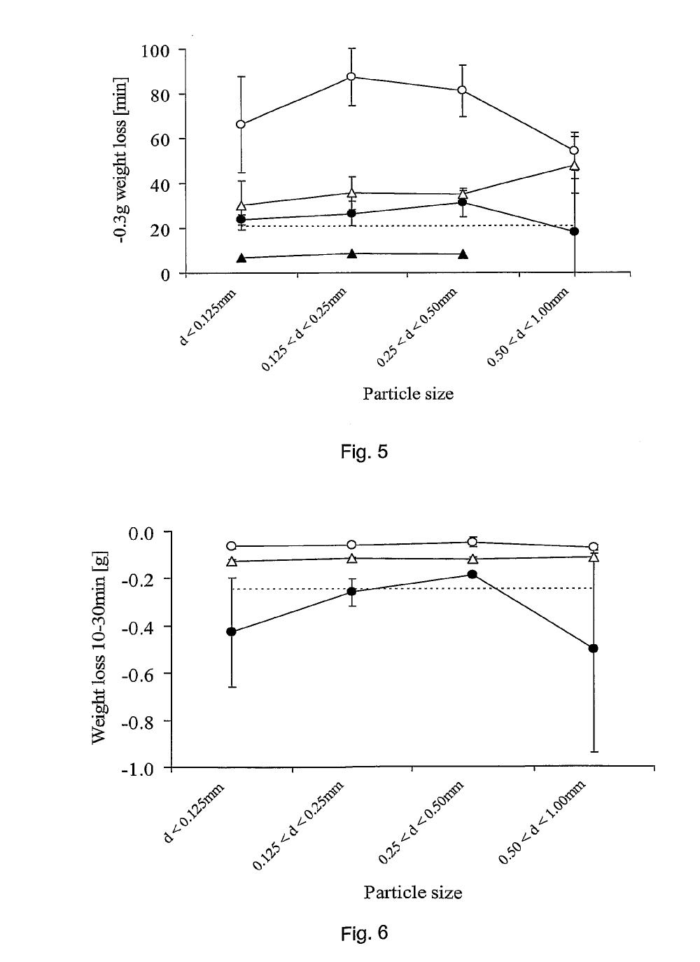

The results obtained with 4 to 5 size fractions of 2 different NaHyA powders are shown in FIGS. 1-4. The results suggest that a particle size larger than 1 mm is not adequate. After autoclaving the samples at 125° C. for 18 min, and drying them at 60° C. for 4 hours, the sample cohesion is decreased due to a decrease of the average MW of NaHyA (FIG. 5-6). Interestingly, the results suggest that the size fraction 0.5-1.0 mm of the 1.76 MDa NaHyA leads to worse results than the other 3 tested fractions. To summarize, these results show that the hydrogel particle diameter should be lower than 1 mm, possibly lower than 0.5 mm.

The invention and further developments of the invention are explained in more detail in the following examples:

### EXAMPLE 1

### A) Obtaining a Sterilized Sodium Hyaluronate

An aqueous solution of NaHyA having a MW of 1428 kDa and a particle diameter of 0.125 to 0.500 mm was autoclaved for 18 minutes at 125° C. By the autoclaving the MW of the NaHyA was reduced from originally 1400 kDa down to 800 to 1000 kDa (as measure by viscosimetry). The reduction of the MW had no negative effect on the qualities. Drying after wet autoclaving was done in dry air in the presence of P₂O₅ powder under sterile conditions. The sterility was provided by two steam-permeable membranes used to package the material before autoclaving.

### B) Obtaining a Bone-Replacement Material of Putty Consistency

0.12 g of the obtained dried NaHyA (according to step A), 1.1 g of β-tricalcium phosphate powder (with a diameter in the range of 0.125-0.500 mm and a specific surface area of 0.01-0.30 m²/g) and 1.1 g of β-tricalcium phosphate powder (with a diameter in the range of 0.500-0.700 mm and a specific surface area of 0.01-0.30 m²/g) were mixed with 2 ml of sterile water with a spatula for 60 second. The β-tricalcium phosphate powders had a porosity of 60%.

Two minutes after the start of mixing, a slightly elastic and kneadable mass was obtained. This paste was then kneaded to form a long and thin “worm” and inserted into a cancellous bone void resulting from a tibial plateau fracture. The void entry was then closed with the periosteum. Two and a half months after surgery, x-ray pictures demonstrated the presence of new bone in the defect and the start of the resorption process of the β-TCP granules. Full weight bearing could again be applied on the tibia.

### EXAMPLE 2

A mixture of 24 g of porous and angular granules of dicalcium phosphate DCP (with an approximate size of 500 micrometers, a sphericity degree of S=3.1 and a specific surface area close to 7 m²/g) and 1.4 g chondroitin sulfate with a MW of 535 kDa was sterilized by autoclaving for 18 minutes at 125° C. Drying after wet autoclaving was done by freeze-drying under sterile conditions.

The sterile dry mixture was mixed with 25 mL of bone marrow aspirated from the pelvic bone of a 10-year old boy. The resulting mixture was kneaded in a sterilized bowl with a sterilized spatula for 1.5 minutes. Two minutes after the start of mixing, a slightly elastic and kneadable mass was obtained. This paste was then inserted into a cyst present in the humorous of the boy. The void entry was then closed with the periosteum. Six weeks after surgery, x-ray pictures demonstrated the presence of new bone in the defect and the start of the resorption process of the DCP granules. No empty void could be detected which could suggest the formation of a new cyst.

### EXAMPLE 3

A mixture of 0.3 g of 0.2-0.3 mm porous and spherical granules of calcium deficient hydroxyapatite having a specific surface area of 30 m²/g) and 0.3 g of 0.5-0.7 mm porous and spherical granules of calcium deficient hydroxyapatite having a specific surface area of 30 m²/g) was mixed with 50 mg of biotechnologically generated hydroxypropylmethyl cellulose with a MW of 900 kDa.

This mixture was sterilized by autoclaving for 18 minutes at 125° C. Drying after wet autoclaving was done by the action of dry air under sterile conditions.

Then, 0.1 mL of 5 weight percent gentamicin sulfate solution were added to the dried mixture and thoroughly mixed for 2 minutes. The resulting kneadable material was highly suitable as a plastic bone-replacement material and as a gentamicin delivery system.

### EXAMPLE 4

0.2 g of sodium alginate (MW=50-500 kDa I particle diameter <0.71 mm) and 2.5 g of spherical granules of carbonated apatite (with a grain size of 200-300 microns and a specific surface area of 80 m²/g) were mixed and sterilized by autoclaving for 18 minutes at 125° C. Drying after wet autoclaving was done by the action of vacuum under sterile conditions.

Then 2.0 g of sterile Ringer solution were stirred into this dried mixture. This resulted in a kneadable material which was able to be used as a plastic bone-replacement material.

### EXAMPLE 5

0.18 g of NaHyA (MW=1.1-1.3 million Dalton), 2.5 g of spherical granules of carbonated apatite (with a grain size of 200-300 microns and a specific surface area of 80 m²/g), 1.0 g of tantalum powder (0.5 mm in diameter) and 1.5 g of porous and angular granules of β-tricalcium phosphate (β-TCP) having a grain size of 125 to 500 micrometers, a sphericity of S=2.5 and a specific surface area in the range of 0.01-0.30 m²/g) were mixed thoroughly and sterilized by autoclaving for 18 minutes at 125° C. After drying of the sterile mixture 0.5 ml of platelet-rich plasma under sterile conditions an amount of 1.5 ml of sterile deionized water were then stirred into this mixture. After thorough mixing, this resulted in an excellent plastic kneadable material which was able to be used as a plastic bone-replacement material.

### EXAMPLE 6

0.18 g of NaHyA (MG=1.1-1.3 million Dalton), 1.0 of porous and angular granulates of β-tricalcium phosphate (with a grain size of 500 to 700 micrometers, a sphericity degree of S=2.9 and a specific surface area of 0.01-0.30 m²/g) and 1.5 g of porous and angular granulates of β-tricalcium phosphate (with a grain size of 125 to 500 micrometers, a sphericity of S=2.5 and a specific surface area of 0.01-0.30 m²/g) were mixed thoroughly and sterilized by autoclaving for 18 minutes at 125° C. After drying of the sterile mixture (under sterile conditions) 2 ml of fresh blood were then stirred into this mixture. After thorough mixing, this resulted in an excellent plastic kneadable material which was able to be used as a plastic bone-replacement material.

### EXAMPLE 7

### A) Manufacture of Powder

A mixture of 6.6 g β-TCP spherical granules with a size of 0.125-0.500 mm and a specific surface area of 0.01-0.30 m²/g)) and 0.27 g NaHyA (MW=1100 kDa) was autoclaved for 18 minutes at 125° C.

To make sure that autoclaving is effective and that the mixture stays sterile after autoclaving, the mixture was packaged twice in a blister package closed with a paper cover. The latter cover is permeable for steam, but not for germs. After drying, the double blister package was packaged in an aluminum peel pouch to prevent humidity to decompose NaHyA during shelf life.

### B) Manufacture of Liquid

6 mL of sterile Ringer solution were filled under aseptic conditions into two blister packages closed with an aluminum-coated membrane. The solution was then gamma irradiated with 25-42 kGray to sterilize it.

### C) Use of the Kit

The product kit consisted of a peel pouch containing the dry component (NaHyA and β-TCP granule) and the wet component. The kit was opened by a nurse in the surgical room. The peel pouch containing the dry component was opened above the sterile surgical table to drop the double-blister package onto the latter table. Afterwards, the surgeon opened both blister packages of the dry component, and placed the second (inner) blister package containing the powder/granule mixture on the sterile surgical table. The nurse opened the double blister containing the solution above the sterile, surgical table and dropped the inner blister onto the table. The surgeon opened the latter blister, poured the liquid into the blister containing the powder/granules, and using a sterile metallic spatula, mixed the two components for one minute. Afterwards, the surgeon took the resulting paste in the fingers and kneaded it.

### EXAMPLE 8

### A) Manufacture of Powder

6.6 g of spherical β-TCP particles [with a diameter of 300+/−50 microns, an apparent density larger than 80% of the theoretical density (3.1 g/cc) and a specific surface area of 0.01-0.30 m²/g] and 0.36 g NaHyA (MW=1429 kDa/particle diameter 0.125 to 0.500 mm) were packaged twice in a humidity-permeable blister and autoclaved for 18 minutes at 125° C. The sample was then freeze-dried until constant weight was reached. The external package was then removed and the inside part (humidity permeable blister) was dropped in a laminar flow bench and packaged in a sterile humidity-impermeable blister.

### B) Manufacture of Liquid

6 mL of sterile distilled water were filled under aseptic conditions into the blister package obtained in step A, and the latter package was closed with an aluminum-coated membrane. The solution was then gamma irradiated with 25-42 kGray to sterilize it.

### C) Use of the Kit

According to example 7.

### EXAMPLE 9

2 g of freezed-dried demineralised cortical allograft bone of particle size ranging from 250-420 microns was added to 93.6 mg of sodium hyaluronate powder (0.1-0.5 mm in diameter; 1.8 MDa molecular weight, 2.7 m³/kg intrinsic viscosity), packaged twice in a steam permeable packaging material, and autoclaved for 18 minutes at 125° C. The sample was then dried for 4 hours at 60° C., and packaged in a steam-proof package. This solid precursor was then mixed with 4.7 mL whole blood from the patient during 2 minutes using a spatula to obtain a malleable putty with excellent formability properties.

1. Precursor for the preparation of a pasty bone replacement material by admixture of a liquid, said precursor comprising
a) a biocompatible substance swellable by the action of water or of an aqueous solution thereby forming a hydrogel; and
b) solid particles made of a substance which is suitable as a bone replacement material, wherein
c) said swellable substance is in the form of discrete particles having a mean dimension in the range of 18 μm to 2000 μm.

2. Precursor according to claim 1, wherein said precursor has been obtained by wet autoclaving and subsequent drying of at least said swellable substance.

3. Precursor according to claim 1, wherein said solid particles are of ceramic or mineral nature.

4. Precursor according to claim 1, wherein said solid particles contain calcium.

5. Precursor according to claim 1, wherein said solid particles are based on demineralized or purified bone material.

6. Precursor according to claim 1, wherein said solid particles are of polymeric nature.

7. Precursor according to claim 1, wherein said solid particles are based on bioglass(es).

8. Precursor according to claim 1, wherein said discrete particles of said swellable substance have a sphericity S smaller than 5.

9. Precursor according to claim 1, wherein said solid particles and said swellable substance are present as a mixture.

10. Precursor according to claim 1, wherein the said swellable substance is in powdered form.

11. Precursor, according to claim 1, wherein said discrete particles of said swellable substance have a mean diameter of at least 18 μm.

12. Precursor, according to claim 1, wherein said discrete particles of said swellable substance have a mean diameter of less than 2000 μm.

13. Precursor according to claim 1, wherein the autoclaving does lead to a decrease of the MW of said swellable substance of minimum 30%.

14. Precursor according to claim 1, wherein the autoclaving does lead to a decrease of the MW of said of said swellable substance of maximum 70%.

15. Precursor according to claim 1, wherein the autoclaving is performed at a temperature in the range of 110° to 140° C.

16. Precursor according to claim 1, wherein said drying is obtained by the action of dry air, vacuum, freeze-drying and/or a desiccating agent.

17. Precursor according to claim 1, wherein the loss on drying at 105° C. of said precursor is smaller than 5%.

18. Precursor according to claim 1, wherein said swellable substance comprises a polyamino-acid or its derivatives.

19. Precursor according to claim 1, wherein said swellable substance comprises one of the following components: a) polysaccharides and their derivatives; b) polylipides, fatty acids and their derivatives; c) nucleotides and their derivatives; d) polymethylenoxide or its derivatives; e) polyethylene, polyethylenoxide or their derivatives; f) polypropylene, polypropylenoxide or their derivatives; g) polyacrylate or its derivatives or a combination of the components as listed in a) through g).

20. Precursor according to claim 1, wherein said swellable substance comprises either a glycosaminoglycane or a proteoglycane or a mixture of those two substances.

21. Precursor according to claim 20, wherein said glycosaminoglycane is a hyaluronic acid, chondroitinsulfate, dermatansulfate, heparansulfate, heparine or keratansulfate.

22. Precursor according to claim 1, wherein said swellable substance is hyaluronic acid or sodium hyaluronate.

23. Precursor according to claim 1, wherein said swellable substance is of biological origin.

24. Precursor according to claim 1, wherein said swellable substance is of fully synthetic origin.

25. Precursor according to claim 1, wherein said swellable substance comprises a biotechnological generated substance.

26. Precursor according to claim 1, wherein the MW of said swellable substance is—after sterilization—larger than 300,000 Dalton.

27. Precursor according to claim 1, wherein the MW of said swellable substance is larger than 1,000,000 Dalton.

28. Precursor according to claim 1, wherein said swellable substance has an intrinsic viscosity greater than 2.0 m³/kg before sterilization.

29. Precursor according to claim 1, wherein swellable substance has an intrinsic viscosity of at least 1.3 m³/kg after sterilization.

30. Precursor according to claim 29, wherein at least 80% of said intrinsic viscosity is reached within 5 minutes after the start of mixing.

31. Precursor according to claim 1, further comprising any drug having an active effect on bone metabolism.

32. Precursor according to claim 1, wherein the said solid particles have at least a partially porous structure.

33. Precursor according to claim 32, wherein the pore size of said solid particles is between 10 nanometers and 500 micrometers.

34. Precursor according to claim 32, wherein the porosity of said solid particles is between 60 and 90 percent.

35. Precursor according to claim 1, wherein the average diameter of said solid particles is between 100 and 500 micrometers.

36. Precursor according to claim 1, wherein said solid particles comprise a calcium phosphate which is characterized by a molar Ca/P relationship between 1.0 and 2.0.

37. Precursor according to claim 1, wherein said solid particles comprise a calcium-phosphate which is characterized by a molar Ca/P relationship between 1.45 and 1.52.

38. Precursor according to claim 36, wherein said calcium phosphate is selected from the following group:
Dicalcium phosphate-dihydrate (CaHPO₄×2H₂O), dicalcium phosphate (CaHPO₄), alpha-tricalcium phosphate (alpha-Ca₃(PO₄)₂), β-tricalcium phosphate (β—Ca₃(PO₄)₂), calcium-deficient hydroxyapatite (Ca₉(PO₄)₅(HPO₄)OH), hydroxyapatite (Ca₁₀(PO₄)₆OH)₂), carbonated apatite (Ca₁₀(PO₄)₃(CO₃)₃(OH)₂), fluoro apatite (Ca₁₀(PO₄)₆(F,OH)₂), chloro apatite (Ca₁₀(PO₄)₆(CI,OH)₂), whitlockite ((Ca,Mg)₃(PO₄)₂), tetracalcium phosphate (Ca₄(PO₄)₂O), oxyapatite (Ca₁₀(PO₄)₆O)I β-calcium pyrophosphate (β—Ca₂(P₂O₇), α-calcium pyrophosphate, gamma-calcium pyrophosphate, and octacalcium phosphate (Ca₈H₂(PO₄)₆×5H₂O), whereby all aforementioned calcium phosphates may be doped with elements selected from the group consisting of Na, Cl, F, S, C, Sr, Mg, Zn, Si, Fe, Li, K and Ag.

39. Precursor according to claim 1, wherein said solid particles comprise a mixture of different calcium phosphates.

40. Precursor according to claim 1, wherein said solid particles comprise a calcium sulfate chosen from the following group: anhydrous—CaSO₄, hemihydrate—CaSO₄.1/2H₂O, dihydrate—CaSO₄.2H₂O, and their polymorphs.

41. Precursor according to claim 1, wherein said solid particles comprise calcite, aragonite or vaterite which are different polymorphs of calcium carbonate.

42. Precursor according to claim 1, wherein said solid particles comprise a mixture of different calcium phosphates, calcium sulfates, calcium carbonates, and/or calcium-containing bioglass.

43. Precursor according to claim 1, wherein the specific gravity of said solid particles is between 0.5 and 1.0 g/ccm.

44. Precursor according to claim 1, wherein said solid particles have a non-spherical shape.

45. Precursor according to claim 1, wherein said solid particles have a specific surface area (SSA) of larger than 0.01 m²/g.

46. Precursor according to claim 1, wherein said solid particles have a specific surface area (SSA) of larger than 5 m²/g.

47. Precursor according to claim 1, wherein said discrete particles of said swellable substance have a mean volume of at least 3·10⁻⁶ mm³.

48. Precursor according to claim 1, wherein said discrete particles of said swellable substance have a mean volume of maximum 4.2 mm³.

49. Precursor according to claim 1, wherein said precursor contains less than 10 weight-percent of gelatin.

50. Precursor according to claim 1, wherein said precursor contains a radiopacifier.

51. Precursor according to claim 50, wherein said radiopacifier is selected from the following group: tantalum powder, tungsten powder, Titanium powder, zirconium oxide powder, bismuth oxide powder, iode-based liquid.

52. Method for preparing the precursor according to claim 1 comprising the following steps:
a) wet autoclaving of said swellable substance avoiding polymerization of said swellable substance;
b) drying of the product obtained in step “a” avoiding polymerization;
c) mixing the product obtained in step “b” with said solid particles.

53. Bone replacement material obtained by admixing a liquid to the precursor according to claim 1.

54. Bone replacement material according to claim 53, wherein said liquid is pure water, sterile demineralized water, an aqueous solution, a sterile saline solution, sterile Ringer solution, serum, blood, bone marrow an antimicrobial drug solution or a solution containing osteoinductive substances and/or drugs against osteoporosis.

55. Bone replacement material according to claim 53, wherein the ratio between the dry weight of the swellable substance and the liquid is in the range of 0.001 and 0.500.

56. Bone replacement material according to claim 55, wherein said ratio is in the range of 0.01 and 0.20.

57. Bone replacement material according to claim 53, wherein the weight relationship between the hydrated hydrogel and the solid particles is larger than 0.2.

58. Bone replacement material according to claim 53, wherein the weight relationship between the hydrated hydrogel and the solid particles is smaller than 4.

59. Kit comprising the precursor according to claim 1 and a liquid suitable for admixing to said precursor in order to convert the resulting mixture into a kneadable mass for bone replacement.

60. Kit according to claim 59, wherein said liquid is pure water, sterile demineralized water, an aqueous solution, a sterile saline solution, sterile Ringer solution, serum, blood, bone marrow, an antimicrobial drug solution or a solution containing osteoinductive substances and/or drugs against osteoporosis.
